(19) United States
(12) Patent Application Publication
(10) Pub. No.: US 2026/0108274 A1
(43) Pub. Date: Apr. 23, 2026

(54) METHODS AND APPARATUS FOR REDUCTION OF MATERNAL INJURY DURING CHILDBIRTH

(71) Applicants: Lisa Molloy, Mountainview, CA (US); Kelly Ashfield, Mountainview, CA (US); Connor Wilson, Aptos, CA (US); Stephen Kmak, Santa Clara, CA (US)

(72) Inventors: Lisa Molloy, Mountainview, CA (US); Kelly Ashfield, Mountainview, CA (US); Connor Wilson, Aptos, CA (US); Stephen Kmak, Santa Clara, CA (US)

(21) Appl. No.: 19/313,614

(22) Filed: Aug. 28, 2025

Related U.S. Application Data
(60) Provisional application No. 63/689,659, filed on Aug. 31, 2024.

(51) Int. Cl. A61B 17/42; A61B 17/00; A61B 90/00; A61M 29/02

(52) U.S. Cl. CPC A61B 17/42; A61M 29/02; A61B 2017/00477; A61B 2017/00526; A61B 2017/00557; A61B 2090/065; A61M 2205/332; A61M 2210/1475

(57) ABSTRACT

Devices and methods that prepare the vaginal tissue of the mother's birth canal during labor so as to prevent pelvic floor damage, and vaginal and perineal lacerations. The devices may be modular and in one example include two modules, a dilator module that comprises a dilator capable of expanding the vaginal canal of a mother who is experiencing labor and a control module that is capable of connecting to the dilator module. The two modules can be separated so that the dilator module can be physically separated from the control module. In one embodiment the dilator module is comprised of disposable elements and maybe disposed of after use. The control module may be a reusable module that can be covered during use and sterilized after use for reuse in subsequent procedures.

### RELATED APPLICATIONS

This application claims priority and the benefit under 35 U.S.C. 119(e) of U.S. Provisional Patent Application Ser. No. 63/689,659 filed Aug. 31, 2024, entitled “METHODS AND APPARATUS FOR REDUCTION OF MATERNAL INJURY DURING CHILDBIRTH”. The above cited application is herein incorporated by reference in its entirety.

### FIELD

Devices and methods are described generally relating to female health and in particular to preparing tissue for childbirth. More specifically, the devices and methods described herein therapeutically treat vaginal tissue during labor so as to prevent maternal injury, such as pelvic floor damage, and vaginal and perineal lacerations.

### BACKGROUND

Approximately 134 million women give birth worldwide each year. About 3 million give birth vaginally in the United States. Approximately 8 out of 10 women who give birth vaginally will experience some degree of vaginal tearing. In the U.S, roughly 1.2 million, or 40%, experience a spontaneous laceration or episiotomy (planned surgical cut for high-risk patients) that requires surgical repair. Between 1.5% and 15% of lacerations are considered severe and require extended healing time and pain management and can result in difficult reparative surgery and a considerable decrease in quality of life measurements. Additionally, 15-35% of women suffer damage to their pelvic floor muscles, where the muscles are being physically pulled from the pelvic bone or over-stretched to the point where they become functionally impaired.

Specific risk factors for perineal lacerations during childbirth have been identified, leading to above-average incidence in patient populations. These risk factors include nulliparity (primigravidity, first pregnancy), short perineal body, instrumental delivery (forceps-assisted delivery, vacuum-assisted delivery), prolonged second stage of labor (>1 hour), epidural analgesia, intrapartum infant factors (birth weight over 4 kg, persistent occipitoposterior position, shoulder dystocia), episiotomy, mediolateral or midline, previous anal sphincter tear, maternal age>30, and Asian ethnicity. In addition to the risk factors listed above, perineal lacerations have been linked with a higher incidence of many pelvic floor disorders such as infection, incontinence and prolapse.

Today, there are techniques such as perineal massage, hot compresses and “hands-on” delivery that can be performed in the hospital and have anecdotally showed promise. Additionally, gourds of increasing size have been used in African medicinal and patient care traditions in an attempt to prepare the tissue for labor. Additionally, the Epi-No device was introduced with the goal of pre-stretching tissue to prevent lacerations. The Epi-No is a small balloon that was intended to be used at home by the pregnant woman, 1-3 months before the baby was due. The goal was to inflate the balloon up to approximately 5 cm, at which time the woman would then practice pushing out the device. A another device is disclosed in US patent publication US2015/0265387 and presents implants and dilators for pelvic treatments including for example treatment of vaginal prolapse.

Finally, the Materna Medical company, the assignee hereof, has introduced into clinical studies an expandable dilator capable of treating a patient during the early stages of Labor through a dilation therapy that reduces the likelihood of injury to the mother. Clinical studies conducted at teaching hospitals have shown excellent results for reducing the likelihood of injury to the patient's vaginal tissue during delivery.

Although the Materna Medical company device shows excellent therapeutic effect, there is a need for improved systems that extend such treatments to patient populations having limited access to cutting edge health care and medical devices. As such there is a need in the art for improvements that make the application of clinically effective techniques such as those in clinical studies discussed above more inclusive and available to patient populations that are traditionally underserved by the medical device industry.

### SUMMARY

A device for improving outcomes for, inter alia, obstetrics, gynecology, urology and neonatal patients by reducing injury during the labor and delivery process.

The devices and methods described herein provide improved access to medical care for mothers going through labor and delivery and in particular provide improved access to devices and methods that prepare the vaginal tissue of the mother's birth canal during labor so as to prevent pelvic floor damage, and vaginal and perineal lacerations. As will be described in more detail herein, the devices may be modular and in one example include two modules, a dilator module that comprises a dilator capable of expanding the vaginal canal of a mother who is experiencing labor and a control module that is capable of connecting to the dilator and controlling that dilator to expand and contract as appropriate, typically slowly and carefully, to provide therapeutic tissue preparation prior to delivery of the baby. The two modules can be separated so that the dilator module can be physically separated from the control module. In one embodiment, a releasable mechanical coupling is configured to releasably connect the control module to the dilation module. Typically, the releasable mechanical coupling allows for facile and rapid releasable coupling by a clinician treating the patient. This may reduce the weight of the device disposed within the vaginal canal of the patient and thereby make it easier for the patient during the labor process, such as when the patient is moving through standard positions for finding comfort during the labor process. In one embodiment the dilator module is comprised of disposable elements and may be disposed of after use. The control module may be a reusable module that can be sterilized after use and reused in subsequent procedures.

In one aspect, the systems and methods disclosed herein include a device for dilating a vaginal canal of a patient during labor, and having a dilator module having a longitudinal axis and being sized to fit in a vaginal canal to traverse the vaginal introitus and extend to a position inferior to the cervix, and having an expandable padded exterior configured to expand radially from a contracted configuration having a first diameter to an expanded configuration having a second larger diameter. The device includes a control module configured to cause the dilator module to expand the padded exterior from the contracted configuration to the expanded configuration, and a releasable mechanical coupling configured to releasably connect the control module to the dilation module.

Optionally, the control module comprises a programmable controller configured to control the control module to expand the padded exterior to contact a tissue wall of the vaginal canal of the patient. Typically, the programmable controller may be a microprocessor, a microcontroller, and application specific integrated circuit, or similar structure of the type having a logic unit and memory. Instructions of the type capable of being implemented by a microprocessor can be loaded into a memory and the programmable controller can direct the control module to operate as directed by such instructions. Typically, the programmable controller can include a circuit board assembly and in some embodiments may be housed within the control module that attaches to the dilator module. In alternate embodiments, the programmable controller may be spaced distant from the dilator.

Optionally, the device may include a force sensor capable of measuring a force applied by the padded exterior to the tissue wall of the vaginal canal of the patient and communicating with the programmable controller.

In some embodiments, the releasable mechanical coupling includes a coupling for delivering a torque to the dilator module to control expansion and contraction of the dilator module. In some embodiments the releasable mechanical coupling includes a fluidic coupling for delivering a fluid under pressure to the dilator module to control expansion and contraction of the dilator module. The fluidic coupling can couple the dilator module to a source of fluid pressure, such as air pressure or some other fluid including water. In such an embodiment, the dilator module may be responsive to a fluid pressure for expanding the dilator module to provide therapeutic pressure and force to the vaginal canal of the patient. The releasable mechanical fluidic coupling can provide for rapid and facile connecting and disconnecting of the dilator module to the control module that controls the flow of fluid to and from the dilator module.

In some embodiments, the wherein the control module is shaped like a handle of the type that a clinician can manipulate during the insertion of the device into the vaginal canal of the patient, the control module may provide at handle having a length that is sized to be sufficiently large to allow the clinician to manipulate the control module as a handle. But optionally, at the same time the control module will be sized such that the handle it provides extends along the longitudinal axis a length that is selected to extend from the vaginal canal of the patient for a distance that is sufficiently short to allow the patient to comfortably carry the device during the typical movements of labor. For example, in some embodiments the handle has a length along the longitudinal axis of between 4 cm and 20 cm. Further, the handle, which may be the exterior shell of the control module, may have a diameter sized to be manually gripped by treating clinician and optionally may be a diameter sized between 2 cm and 10 cm.

In some embodiments, the control module includes a processor for implementing a program to automatically expand the dilator module. Thus the control module can implement a program of therapeutic expansion of the dilator for providing a program of therapy for the patient during labor. Typically, the program of expansion takes place during the first stage of Labor and is completed prior to the second stage of Labor.

In some embodiments, the dilator module releasably connects to the control module to align along the longitudinal axis such that a linear force applied to the handle will act to move the dilator module along the longitudinal axis for insertion into the vaginal canal of the patient.

In other aspects, the systems and methods described herein include methods for dilating a vaginal canal of a patient during labor, comprising providing a dilator module having a longitudinal axis and being sized to fit in a vaginal canal to traverse the vaginal introitus and extend to a position inferior to the cervix, and having an expandable padded exterior configured to expand radially from a contracted configuration having a first diameter to an expanded configuration having a second larger diameter. The method may also provide a control module configured to cause the dilator module to expand the padded exterior from the contracted configuration to the expanded configuration, and provide a releasable mechanical coupling configured to releasably connect the control module to the dilator module, and for releasably connecting the dilator module to the control module to align along the longitudinal axis.

In some practices, the methods further include applying a linear force to the handle to move the dilator module along the longitudinal axis for insertion into the vaginal canal of the patient. The methods may further comprise controlling the control module to expand the padded exterior to contact a tissue wall of the vaginal canal of the patient. Optionally, controlling includes implementing a program to automatically expand the dilator module to therapeutically treat tissue of a vaginal canal during a first stage of labor. Further optionally, controlling includes pausing the program of automatically expanding. For example, the implemented program may provide for expansion of the dilator for a select period of time, such as some number of seconds. After the select period of time and expansion has taken place, the implemented program may pause expansion for again a set period of time period during this sort of pause, the tissue walls given a period of time to relax and adjust to the applied force and the increased diameter of the vaginal canal. After a programmatic period of pause, the system may then under the control of the implemented program expand the dilator to provide additional force and expansion of the vaginal canal.

In some practices the methods further include operating the releasable mechanical coupling to release the control module from the dilator module during stage one of labor.

It will be apparent to those of skill in the art that by including in the device a reusable control module, the cost of the device may be reduced as it allows for multiple procedures to use the same control module. This in turn can reduce the cost of providing therapy to patients. Additionally, it will be understood by those of skill in the art that it is not uncommon that a clinic supporting patients through labor and delivery have multiple patients going through labor and delivery processes at the same time, or substantially the same time. The modular devices described herein may include kits that have multiple sets of dilation modules, each of which is to be used once with the patient and procedure and then disposed of. A single control module may be used multiple times with multiple different dilation modules each dilation module acting on a separate patient. In this way, broader access to the birth canal dilation therapies described herein may be provided as the dilation procedure for each patient may involve a separate dilation module, but only a single control module may be needed during these multiple procedures. It will be further understood by those of skill in the art, that having a removable control module allows for the patient to only carry the dilation module within the vaginal canal. This eliminates from the procedure requiring the patient to carry the weight of both the dilation module and the control module during the activities of labor. As a patient typically will wish to move about the birthing bed, or move across the room, and often undertake certain therapeutic exercises that can alleviate discomfort during the labor process, the dilation device may move and may fall from the patient. By having a removable control module, a clinician is able to remove the control module from the dilation module, and thus the vaginal canal of the patient need only support the weight of the dilation module in order to keep the dilation module in place as the patient undertakes certain activities such as moving about the room or repositioning themselves to reduce discomfort.

Typically, the control module is shaped as a handle that is sized to be manipulated by the treating clinician. The dilator module attaches to the control module so that both modules align along a longitudinal axis. In preparation for treatment, a clinician can insert the dilator module into the vaginal canal of the patient by inserting the dilator module across the vaginal introitus and applying a linear force to the handle so that the dilator module and control module move forward toward the cervix along a path generally aligned with the longitudinal axis.

In one embodiment the device for dilating a vaginal canal of a patient during labor includes a dilation device that has a longitudinal axis and is sized to fit in the vaginal canal to traverse the vaginal introitus and extend to a position inferior to the cervix. An expandable padded exterior is provided and the dilation device is configured to expand radially from a contracted configuration having a first diameter to an expanded configuration having a second larger diameter. The device further includes a handle that typically has an actuation mechanism configured to cause the dilation device to expand the padded exterior from the contracted configuration to the expanded configuration and a releasable mechanical coupling configured to connect the actuation mechanism to the dilation device and to align the handle along the longitudinal access of the dilation device such that a linear force applied to the handle will act to move the dilation device into the vaginal canal of the patient.

Typically, there will be a controller that is part of the control module and is configured to control the expansion module to expand the padded exterior to contact a tissue wall of the vaginal canal of the patient. Optionally, the device includes a force sensor that is capable of measuring the force applied by the padded exterior to the tissue wall of the vaginal canal of the patient. The force sensor typically communicates the measured applied force to the controller. In this way the controller can respond to the pressure applied by the padded exterior to the tissue wall of the vaginal canal.

Optionally, the releasable mechanical coupling includes a rotating coupling for delivering a torque to the dilation module to drive and control expansion and contraction of the dilation module.

In some embodiments the handle of the device has a length along the longitudinal axis selected to extend from the vaginal canal of the patient for a length that is sufficiently short to allow the patient to comfortably carry the device during the typical movements of Labor. In some embodiments the handle has a length along the longitudinal axis of between 4 cm and 20 cm. Typically, the diameter of the handle is sized to be manually gripped by a treating clinician who is holding the handle to manipulate the device, such as by applying a linear force onto the handle to move the dilation module along the longitudinal axis and into place within the vaginal canal of the patient.

In another aspect, the systems and methods described herein include a method of manufacturing a device for dilating a vaginal canal of a patient during labor, that includes providing a dilator module having a longitudinal axis and being sized to fit in a vaginal canal to traverse the vaginal introitus and extend to a position inferior to the cervix, and having an expandable exterior configured to expand radially from a contracted configuration having a first diameter to an expanded configuration having a second larger diameter. Providing an control module configured to cause the dilator module to expand the exterior from the contracted configuration to the expanded configuration, and attaching to at least one of the control module and the dilator module, a releasable mechanical coupling configured to releasably connect the control module to the dilator module.

Some embodiments of the device can stretch the perineal tissue until the diameter of the vaginal introitus has reached a target diameter of approximately 8 cm to 10 cm, roughly the size of the fetal head. In some embodiments, the dilator penetrates the first 3-4 cm of the vagina, the introitus, and gradually expands the vagina from a resting diameter of 2-3 cm to a fully expanded diameter equal to the size of the delivering fetus, approximately 8 cm to 10 cm.

The devices described herein can be inserted during the first phase of labor and removed just prior to the second phase of labor, allowing the rest of the birthing process to proceed as normal. The device is intended to be used prior to the second phase of labor to prevent a baby from contacting the device during delivery. Depending on how long it takes to achieve full vaginal dilation to approximately 10 cm, the device can be inserted early during the first phase of labor with only very minor amounts of dilation. The dilation diameter target of 10 cm means that the device should be used in a hospital under the supervision of trained obstetricians and nurses. The device can be used without any anesthesia, or under local anesthetic. The tissue can also be prepared after administering an epidural, which would eliminate any pain or discomfort the device may cause. Local anesthesia can also be placed on the tissue contacting surfaces of the device to minimize pain.

### BRIEF DESCRIPTION OF THE DRAWINGS

The foregoing and other objects and advantages of the invention will be appreciated more fully from the following further description thereof, with reference to the accompanying drawings wherein;

FIGS. 1A-1C illustrate a device as described herein for careful preparation of stage two delivery.

FIG. 2 is one view of a device of the type described herein.

FIGS. 3A and 3B show two modules of a device described herein and in a separated configuration.

FIG. 4 depicts a cross-sectional view of a device having two modules that are connected for dilation.

FIGS. 5A-5C depicts in more detail a coupling between a dilator module and an expansion module.

FIG. 6 is a pictorial representation of a cut away view of a device as described herein.

FIG. 7 shows a control module of a device such as the device depicted in FIG. 5.

FIGS. 8A and 8B show in more detail a dilator module of a device such as the device depicted in FIG. 5.

FIG. 9 illustrates an example of the control module operating the dilator module.

FIG. 10 illustrates an example of a dilation process for preparing the patient for stage II of labor and delivery.

FIGS. 11A-11D depict several common movements of a patient during Labor.

FIG. 12 depicts a kit having several dilator modules and one actuation module.

FIG. 13 depicts a further embodiment of the systems and methods described herein.

### DETAILED DESCRIPTION

To provide an overall understanding of the devices and methods described herein, certain illustrative embodiments will now be described, including a modular device for therapeutically treating tissue during labor to reduce maternal injury during childbirth, as well as methods for using and manufacturing the same. However, it will be understood by one of ordinary skill in the art that the devices and methods described herein can be adapted and modified to realize other embodiments and that such other additions and modifications and resulting embodiments will not depart from the scope hereof.

The devices and methods described herein provide improved access to medical care for mothers going through labor and delivery and in particular provide improved access to devices and methods that prepare the vaginal tissue of the mother's birth canal during labor so as to prevent pelvic floor damage, and vaginal and perineal lacerations. As will be described in more detail herein, the devices may be modular and in one example include two modules, a dilator module that comprises a dilator capable of expanding the vaginal canal of a mother who is experiencing labor and a control module that connects to the dilator module by a releasable mechanical coupling and controls that dilator module to expand and contract as appropriate, typically slowly and carefully, to provide therapeutic tissue preparation prior to delivery of the baby. The two modules can be separated so that the dilator module can be separated from the control and drive module. In one embodiment the dilator module is comprised of disposable elements and maybe disposed after use. The control and drive module may be a reusable module that can be sterilized after use, maintained sterile, such as by using disposable plastic covers over the control module, like with dental tools. Alternatively, it may be that the control module is configured to be kept outside the field of sterility. In both cases, the control module may be reused in subsequent procedures.

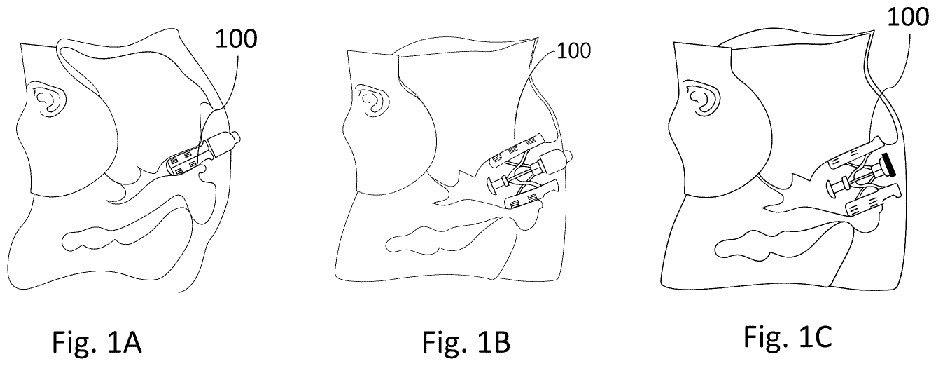

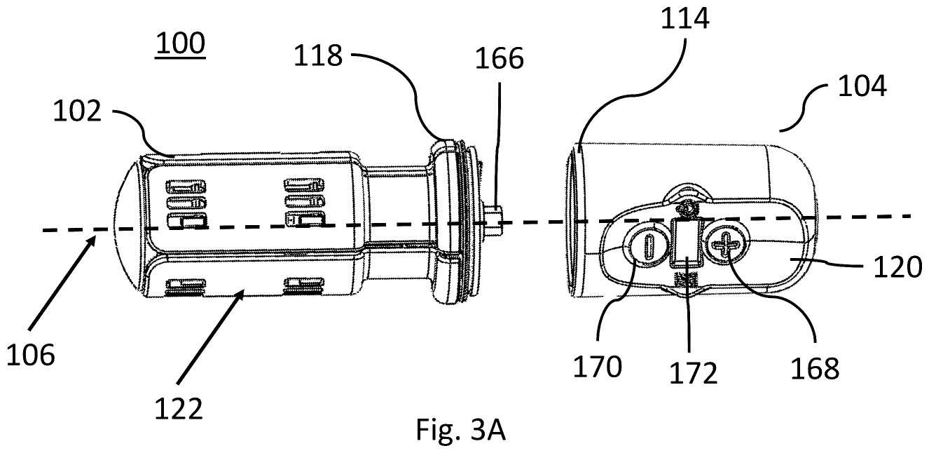

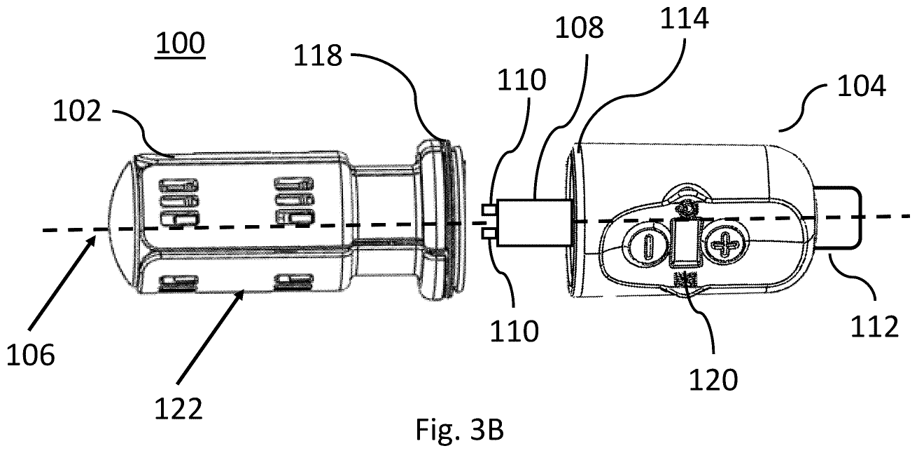

FIGS. 1A-1C illustrate a medical device as described herein for careful preparation of vaginal tissue for reducing risk of maternal injury during the delivery process. More particularly, FIGS. 1A-1C illustrate one embodiment of a vaginal dilation device 100 of the type described herein. In FIG. 1A the device 100 is disposed within the vaginal canal of a patient and the device 100 is in a contracted configuration, which means it has a diameter suited for insertion into the patient without harm or significant discomfort. As will be described in more detail below, the device 100 may include two parts a dilation module and a control module. These two modules can be separated as depicted more fully in FIGS. 3A and 3B. In any case, FIG. 1B depicts the device 100 disposed within the vaginal canal of the patient similar to FIG. 1A however in FIG. 1B the device 100 is in an expanded or at least partially expanded configuration. In either case, it can be seen that in FIG. 1B the device 100 has expanded in diameter such that it is in contact with and is pressing up against the tissue wall of the vaginal canal of the patient and acts to therapeutically treat the tissue with careful and controlled application of force to increasing the diameter of the vaginal canal of the patient. FIG. 1C depicts the expanded, or perhaps partially expanded, dilation device 100 but in this depiction, the dilation module is separated from the control module. Detachment of the dilation module from the control module may be done for any suitable reason, including increasing patient comfort and for reducing weight of the device 100 so as to reduce the likelihood of the device 100 slipping from the patient. FIGS. 1A through 1C depict that the device 100 is sized to fit within the vaginal canal of the patient such that it passes the vaginal introitus and extends into the vaginal canal but inferior to the cervix. The diameter of the device 100 can be altered from a contracted configuration that can have a diameter of three to six centimeters (3 cm to 6 cm) for example to an expanded diameter of typically about ten centimeters (8-10 cm). The contracted configuration and the expanded configuration are selected so that during an early stage of Labor the device 100 in a contracted configuration can be relatively easily and comfortably inserted into the vaginal canal of the patient. Once so inserted the device 100 may be expanded and that expansion will typically increase until the diameter of the device is proximate to the expected diameter the vaginal canal should attain once the cervix opens and the baby begins passing through the vaginal canal.

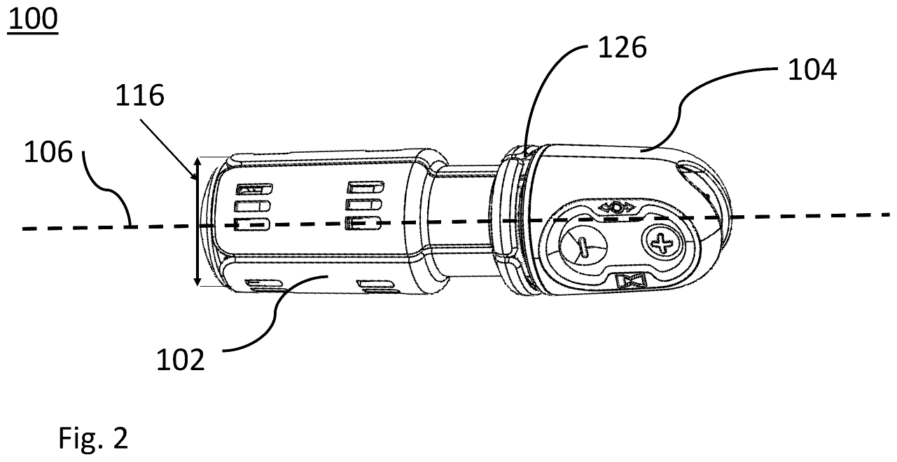

FIG. 2 depicts one embodiment of the medical devices described herein. FIG. 2 depicts a modular dilation device 100 that includes a dilator module 102 and a control module 104. The dilator module 102 and the control module 104 are connected by a releasable mechanical coupling 126. In FIG. 2 the device 100 is shown with the dilator module 102 in a contracted configuration have a diameter 116 sized to fit in the vaginal canal of a relaxed patient. As shown in FIG. 2, the dilator module 102 and the control module 104 are aligned along a longitudinal axis 106 shown as a dashed line extending through the dilation device 100. In the embodiment depicted in FIG. 2 the control module 104 is shaped and sized as a handle. A clinician can grip the control module 104 for inserting the dilator module 102 into the vaginal canal of the patient. To that end, the clinician may apply a linear force against the control module 104 to move the dilator module 102 along the longitudinal axis 106 which may be aligned with the lumen formed by the vaginal canal of the patient. The dilator module 102 may be sized and shaped to fit within the vaginal canal of the patient so that it extends from the vaginal introitus to a position inferior to the cervix.

FIG. 3A depicts one embodiment of the device 100. FIG. 3A shows this embodiment of device 100 in a separated configuration wherein the dilator module 102 is physically separated from the control module 104. FIG. 3A shows that this embodiment of the dilator module 102 has a padded exterior 122 that consists of expandable arms that carry light and thin pads of a soft pliable plastic material suitable for engaging against the tissue wall of the patient. FIG. 3A further shows that the dilator module 102 has an exterior coupling collar 118 at its proximal end. The exterior coupling collar 118 forms part of the releasable mechanical coupling 126 shown in FIG. 2 that releasably connects the dilator module 102 with the control module 104. The control module 104 includes an interior coupling collar 114. The exterior coupling collar 118 can slide into and fit about the interior coupling collar 114. In one embodiment, the collars 114 and 118 each have a partially threaded ring that oppose each other, so that one threaded ring can screw into engagement against the other threaded ring. Optionally, one collar includes one or more detents and the other collar includes one or more cooperating recesses. The detents and recesses are arranged on the respective collars 114 and 118 so that the collars 114 and 118 can rotate into engagement such that a detent can snap into a cooperating recess to provide a mechanical lock to hold the dilator module 102 into a select engagement with the control module 104. Typically, the collars 114 and 118 can be rotated manually in a one direction, for example clock-wise, to cause the collars to join together and lock in place, and a manual force provided in an opposite, for example counter-clockwise, direction can rotate the detents out of the respective recesses to allow disengagement of the collars 114 and 116 and release of the dilator module 102 from the control module 104. Such threaded collars can thereby form one embodiment of a releasable mechanical coupling such as the depicted releasable mechanical coupling 126 of FIG. 2. This is one embodiment of a releasable mechanical coupling 126 and other embodiments may be used, such as for example releasable mechanical coupling 126 that includes direct snap fit couplings that do not use threaded couplings. Other embodiments include bolt connectors that include bolts and cooperating threaded receptacles that allow one module, such as the dilator module 102, to carry one or more bolts that can attach to the dilator module 102 and can engage with threaded receptacles in the control module 104 to allow the dilator module 102 to bolt to the control module 104. The bolts can be removed to release the two modules, allowing the control module to be reused. Other embodiments for the releasable mechanical coupling 126 suited to join the modules 102 and 104 may be used and the type of mechanism used may be selected based on the modules being used and the circumstances around the reuse of the control module 102.

FIG. 3A shows an embodiment having a removable battery 112 that can be slidably engaged to the device 100. In other embodiments, the battery 112 may be embedded into the control module 104 such that it is fully housed within the control module 104. The option selected for housing the battery or having the battery be removable may depend on different factors, such as sterilization methods recommended for sterilizing the control module 104 after use.

In the embodiment of FIG. 3A, the dilator module 102 includes a drive coupling 166 that extends from the proximal end of the dilator module 102. The drive coupling 166 is sized to extend into the interior of the control module 104 when the dilator module 102 is connected to the control module 104. The drive coupling 166 may be configured to seat within a drive shaft that is disposed within the control module 104. When the drive coupling 166 is coupled to that drive shaft, the drive shaft can transmit rotational force (torque) from the drive shaft to cause the drive coupling 166 to rotate. Rotation of the drive coupling 166 can cause the dilator module 102 to expand from a contracted configuration to an expanded configuration and contract from that expanded configuration back to the contracted configuration. The control module 104 includes a control interface 120. The control interface 120 includes an expansion button 168, a contraction button 170, and a digital readout 172. The control interface 120 provides a user interface that can be manually operated by a clinician. The clinician can use the expansion button 168 to direct the control module 104 to cause the drive shaft to drive the coupling 166 2 expand the dilator module 102. Similarly, the clinician can activate the button 170 to cause the drive shaft of the control module 104 to rotate the coupling 166 to contract the dilator module 102 from an expanded configuration into a contracted configuration. The digital readout 172 can present to the clinician information such as the radial circumference of the dilated module for its particular state of expansion.

FIG. 3B depicts an alternate embodiment, wherein the control module 104 includes a spindle 108 at its distal end, with two detents 110, an interior coupling collar 114, a control interface 120 and a removable battery 112. As depicted in FIG. 3B, the spindle 108 is aligned along the longitudinal axis 106 such that the spindle 108 can be can be pushed forward along the axis 106 and into engagement with the dilator module 102. In some embodiments that are motorized, the spindle 108 may be connected to a motor and maintained within the control module 104. In other embodiments that are manually operated, the spindle 108 may connect to a manually operated assembly, often a rotatable dial at the proximal end of the control module 104, that the clinician can turn to cause the spindle 108 to rotate. The manually operated assembly can include a ratchet assembly, with pawls and detents. The ratchet assembly rotates under the force of the clinician's manual twisting of a handle, to allow for expansion in incremental steps thereby providing ratcheting increments for the expansion of the distal module.

In both embodiments, the motor or the manual rotation of the dial may rotatably drive the spindle 108 to create a torque force that may be used to expand and contract the dilator module 102 from a contracted configuration to an expanded configuration. The control module 104 may carefully and typically incrementally, expand the dilator module 102 to provide a therapeutic force selected to slowly and carefully allow the tissue wall of the vaginal canal to stretch to a medically selected diameter, typical of the end point diameter of the vaginal canal during delivery of the baby. Optionally, the device may include a force sensor capable of measuring the force applied by the padded exterior of the arms to the tissue wall of the vaginal canal of the patient. The force sensor may be a sensor component of the type commonly used by electrical instruments to measure forces. It may also be a sensor that measures the current load taken up by the motor 142 and translates that load into a measure of applied force. In both cases, the force is communicated to the motor control board (and its programmable controller) for use by the device to act as a safety cut off if the force goes above a threshold value.

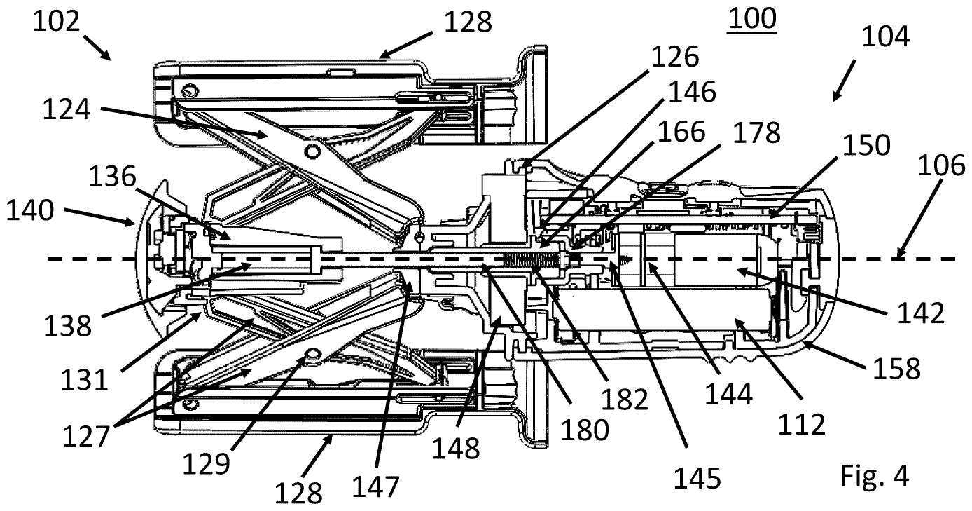

FIG. 4 depicts a cross-sectional view of one embodiment of the medical device 100 to show in more detail the internal elements of the two modules, the dilator module 102 and the control 104. In FIG. 4, the control module 104 and the dilator module 102 are connected together by the releasable connector 126 and configured for the control module 104 to drive the dilator module 102 and cause the dilator module 102 to radially expand and contract.

The embodiment depicted in FIG. 4 corresponds to the embodiment depicted in FIG. 3A that includes a dilator module 102 that includes a drive coupling 166 at its proximal end. The dilator module 102 in the depicted embodiment includes a scissor mechanism 124 that includes several padded arms 128, with FIG. 4 depicting two arms 128, that expand radially outwardly from the longitudinal axis 106. The dilator module 102 further includes a distal contact plate 140, a sled 136, and a screw channel 138 that surrounds the distal end of the threaded screw rod 180. The distal contact plate 140 acts as a fetal head descent detector wherein contact with the fetal head may be detected due to the head contacting the distal contact plate 140. The distal contact plate 140 may be a mechanical switch that depresses along the longitudinal axis 106 in a direction toward the control module 104. Depressing the distal contact plate 140 may cause the scissor mechanism 124 to contract, thereby allowing the dilator module 102 to remove easily from the vaginal canal and away from the descending child. Optionally, depressing the distal contact plate 140 may activate a switch that sends an electric signal to the control module 104 and causes the control module 104 to stop expansion of the dilator module 102 and optionally causes the control module 104 to contract radially the dilator module 102.

The depicted scissor mechanism 124 includes two scissor arms 127 that are joined at a pin 129. One end of one scissor arm 127 joins to the sled 136 at location 131. In this way, as the threaded screw rod 180 is rotated, for example clock-wise, to travel proximally the threaded screw rod 180 pulls the sled 136 proximally causing the scissor mechanism 124 to expand radially. Alternatively, rotation of the threaded screw rod 180 such that the threaded screw rod 180 moves distally, allows the threaded screw rod 180 to travel distally within the screw channel 138 and thereby allow the arms 124 to contract radially.

The padded arms 128 can include rigid or semi-rigid pads. In the embodiment of FIGS. 1-4, the dilator module 102 includes four sets of padded arms. In other embodiments, any number of pads and arms can be used. For example, one embodiment comprises as few as two or three sets of pads and arms, and other embodiments can include more than four sets of pads and arms, such as five, six, seven or eight or more sets of pads and arms. The sets of pads and arms can be arranged symmetrically or asymmetrically around a central axis of the device.

The pads can be coupled to arms 128 of the scissor-like assembly 124, which can be coupled to a central rod 180 with a threaded screw end 182, that extends along the longitudinal axis 106 of the device 100 and is coupled to the control module 104. The tissue contacting pads can be designed to maintain stability through all dilation diameters. In some embodiments, the pads can have a saddle shape that maintains a constant waist size and pitch when expanded. These pads can be over molded with a compliant biocompatible elastomeric material to help evenly distribute force against the tissue and prevent trauma. Additional pad shapes and designs will be discussed below.

In the embodiment shown in FIG. 4, the padded arms 128 are part of a scissor-like assembly 124 and the padded arms 128 expand outwardly or contract inwardly based on the rotation of the threaded screw rod 180. Since the padded arms 128 are carried on a scissor-like assembly 124, the padded arms 128 can remain parallel to the device 100 and to one another during expansion, which maintains the orientation of the padded arms 128 to the tissue stable during dilation. Additionally, maintaining the pads in a parallel orientation can help keep the device 100, or the dilator module 102, in place against the tissue while maximizing force distribution along the padded arms 128. In optional embodiments, the padded arms 128 may connect to the scissor-like assembly 124 such that the padded arms 128 expand in a non-parallel orientation so that the further the padded arms 128 are expanded from the central axis 106, the more the padded arms 128 are engaged against the vaginal canal and anchored within the vaginal canal.

FIG. 4 also depicts a cross-sectional view of the control module 104. The control 104 includes a motor 142, a clutch assembly 144, a motor control assembly 150, and a bearing channel 148. Additionally, FIG. 4 shows a removable battery 112 that is housed within the control module 104 and connected to the motor control assembly 150. Optionally, the removable battery 112 may be insertable from outside the control module 104 and placed into connection by insertion through a port in the housing 158, in a configuration such as that depicted in the embodiment of FIG. 3B.

In operation, the motor control assembly 150 may respond to user controls to drive the motor 142 to rotate and generate a torque. The user controls, in one embodiment, may be controls such as the controls 120 depicted in FIG. 3B. The motor 142 drives clutch mechanism 144 which in turn drives the drive shaft 145. The motor 142 may be any suitable motor, and typically is an electric motor. In this embodiment the motor 142 is an electric motor and is battery powered, by battery 112. The clutch assembly 144 acts as a clutch that limits the amount of torque that is transferred from the motor 142 to the dilator module 102. The clutch assembly 144 may also put in place a gear ratio that slows the rotational speed of the spindle 108 in comparison to the rotational speed of the motor 142. The clutch 144 is positioned between the motor 142 and a sealing plate 146 (shown partially in FIG. 4). The sealing plate 146 provides a barrier wall that reduces the passing of biological material and sterilization agents into the interior chamber formed by the shell 158 of the control module 104. The sealing plate 146 is typically made from a biocompatible material. Additionally there can be a gasket that seals about the drive coupling 166 to allow the drive coupling 166 to rotate without passing material from the dilator module 102 to the control module 104. The shell 158 is generally made of a plastic material of the type can be sterilized using conventional and accepted techniques for sterilizing components. This can include sink rinses, autoclaves and other known sterilization techniques for medical instruments. Further, another sealing plate 147 can be placed above the threaded screw 180 for the purpose of preventing fluids from traveling along the rotating threaded screw 180 and into the control module 104. Further additionally, the releasable coupling mechanism 126 may include a seal that allows for a releasable connection which is also sealed against movement of biological materials and sterilizing agents and other fluids from outside of the device 100 to the interior of the control module 104. These seals can include biologically compatible materials such as silicone, that provide robust seals that are sufficiently flexible to allow a compressed engagement and seals and thereby prevent easy movement of fluids from one side of the seal to the other.

The motor 142, through optional clutch 144, rotates the drive shaft 145. As depicted in FIG. 4 the drive shaft 145 is shaped to receive the drive coupling 166. The drive shaft 145 at its distal end can have shape that is configured to receive a complementary shaped drive coupling 166. For example, the distal end of the drive shaft 144 may have a hexagonal shaped recess that can receive a hexagonal drive coupling 166. These complementary shapes provide for a mechanical engagement between the drive shaft 145 and the drive coupling 166 that allows the drive shaft 145 to rotate the drive coupling 166 and the by applying a torque to the drive coupling 166. Rotation of the drive shaft 145 will cause rotation of the drive coupling 166. As depicted in FIG. 4 the drive coupling 166 has an interior threaded passage that receives the threaded end 182 of the threaded screw 180. Rotation of the drive coupling 166 by the drive shaft 145 causes the threaded screw 180 to move proximally or distally along the axis 106 depending on the rotation of the threaded end 182 within the interior threaded passage of the drive coupling 166.

The torque applied to the drive coupling 166 causes the threaded rod 180 to move proximally and distally and thereby apply an axial directed force to the sled 136 that will pull the sled 136 proximally or release the sled 136 for distal movement. This in turn will cause the arms 124 to radially expand in response to the axial pull of the threaded screw rod 180 or to contract in response to force from the tissue of the vaginal canal pushing the padded arms 128 radially inward while the threaded screw rod 180 moves distally in channel 138, thereby allowing the scissor mechanisms 124 to contract.

The motor control assembly 150 in this embodiment is housed within the control module 104. In one embodiment, the motor control assembly 150 comprises a checkerboard assembly including a microcontroller, ASIC or other programmable device. The motor control assembly 150 can operate the motor 142 in response to, for example, user controls entered through a user control interface such as the interface 120 depicted in FIGS. 3A and 3B. In some embodiments, the motor control assembly 150 implements a program of therapy for therapeutic expansion of the dilator module 102 whereby incremental expansion of the birth canal takes place over a selected period of time through selected increments with careful application of force. Optionally, the motor control assembly 150 can have a sensor that detects a signal from the distal contact plate 140 that indicates a force on the distal end of the dilator module 102. In response, the motor control assembly can cause the motor 142 to rotate in a direction that provides for radial contraction of the dilator module 102, or take other steps to address the detected force, such as presenting on the user interface an indicator that a force has been detected by the distal contact plate 140. Further, the motor control assembly 150 can include a memory of the type used for storing data. In one embodiment, the motor control assembly 150 keeps track of current expansion of a particular dilator module 102 by storing a parameter representative of the current radial expansion of the dilator module 102 in the memory of the motor control assembly 150. In this way, if the control module 104 is disconnected from the dilator module 102 during a procedure, the control module when reconnected to the dilator module 102 can access a stored a parameter representing the expansion of the dilator module 102 at the time of disconnecting the control module 104 from that dilator module 102.

In an alternate embodiment, such as the embodiment depicted in FIG. 3B, the control module 104 may include a spindle 108 with detents 110 that can slide into the dilator module 102 to fit into receptacles configured to receive the detents 110. The torque is applied along the spindle 108 which drives the threaded rod screw 180. The threaded rod screw 180 will turn within a threaded screw channel causing the sled 136 to move along the longitudinal axis 106 either proximally or distally (toward or away from) the control module 104 depending upon the direction of rotation of the screw. As the sled 136 moves along the longitudinal axis 106, the padded arms of the scissor mechanisms 128 will contract or expand moving radially toward or away from the longitudinal axis 106.

The spindle 108 may be driven by the motor 142 through the clutch assembly 144. The clutch assembly 144 acts as a clutch that limits the amount of torque that is transferred from the motor 142 to the dilator module 102. The clutch assembly 144 may also put in place a gear ratio that slows the rotational speed of the spindle 108 in comparison to the rotational speed of the motor 142. As with the earlier described embodiment, the clutch 144 may be positioned between the motor 142 and a sealing plate 146. The sealing plate 146 provides a barrier wall that reduces the passing of biological material and sterilization agents into the interior chamber formed by the shell 158 of the control 104.

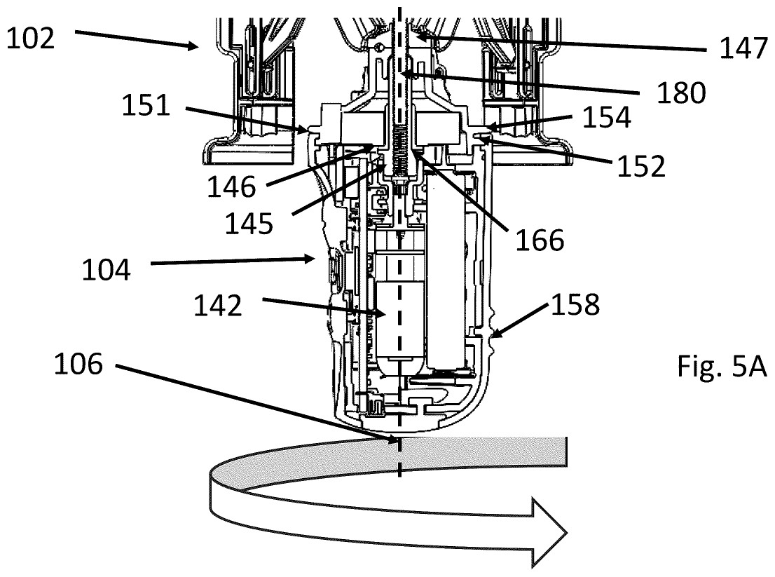

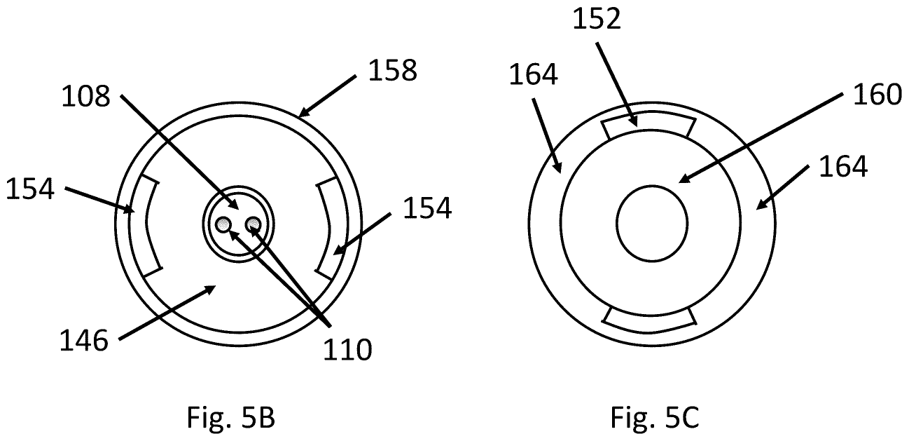

FIG. 5A depicts in more detail one embodiment of a releasable mechanical coupling that releasably connects the control module 102 to the dilator module 104. In this embodiment, the control module 104 has a drive shaft 145 and the dilator has a drive coupling comparable to the embodiment of FIG. 3A. As shown in FIG. 5A, in this embodiment, the releasable mechanical coupling 151 includes complementary collars that interlock to join the dilator module 102 to the control module 104. The collars 154 and 152 interlock, typically by manual rotation of the control module 104 relative to the dilator module 102. The collars 152 and 154, as discussed above, may include detents and recesses for a mechanical fit that reduces likelihood of unwanted rotation and release. Additionally, the detents and recesses may provide for selected alignment of the dilator module 102 with the control module 104. As shown in FIG. 5A, once connected, the drive shaft 145 is connected to the drive coupling 166, both of which are aligned along axis 106. As noted above, seals, such as seal 146 and seal 147, reduce the transfer of material into the control module 104, thereby allowing the control module 104 to be reused. Typically, the control module 104 is covered with a disposable sterile covering, often an elastic covering of material resistant to biological material. This further allows for reuse of the control module 104.

FIGS. 5B and 5C depict plane views of one embodiment of a mechanical coupling that provides a releasable connection between the control module 104 and the dilator module 102. In this embodiment of the type depicted in FIG. 3B, the control module 104 includes one or more collar walls 154 that attach to the distal edge of the interior wall of the dilation module shell 158. In one embodiment each of the collar walls 154 extends around about one quarter of the interior peripheral edge of the shell 158. The dilator module 102 may have a dilator module collar wall 152 that extends proximally from the dilator module 102 and is sized to fit snugly against the interior wall of the shell 158 when in place. In this example embodiment, each of the collar walls 152 may be sized to extend round about one quarter of the interior peripheral wall of the shell 158.

FIG. 5B shows a plane view of the collar wall 154 that extends, at two places, along about one quarter to about one half, collectively between the two depicted walls, of the interior peripheral wall of the shell 158. In alternate embodiments, the collar wall 154 can extend over more of the interior peripheral wall of the shell 158. The collar walls are spaced away from the seal 146, and may be distal to the seal 146, as in the embodiment of FIG. 5A. FIG. 5C depicts by a plane view the complementary collar wall 152 of the dilator module 102. The collar wall 152 attaches to and is supported by a plate 160 that extends proximally from the dilator module 102 and can optionally engage against the seal 146. Optionally, the collar wall 152 can be sized to fit snugly against the interior peripheral wall of the shell 158. On either side of the collar walls 152 are openings 164 that are large enough to receive the complementary collar walls 154.

To connect the control module 104 to the dilator module 102, the user may slide the shell 158 along the longitudinal axis 106 so that the expansion module collar walls 154 pass through the openings 164 presented by the absence of a distal module collar walls 152. As mentioned, the distal module collar wall 152 typically only extends around about one quarter of the circumference of the inner wall of the shell 158. As in this embodiment, there are typically two collar walls 152, this leaves half of the circumference of the interior wall open, and provides space for the collar walls 154 to slide past the collar wall 152. Once slid in place, the shell 158 may be rotated about axis 106 similar to that depicted by the rotation arrow for the embodiment of FIG. 5A. This rotation may turn the expansion module collar walls 154 into a position that engages the collar walls 154 against the complementary collar walls 152 and frictionally is then held in place by contact with the dilator module collar walls 152. This allows for facile assembly of the dilator module with the control modules. The clinician in operation will slide the shell 158 into contact with the dilator module 152, and by manually rotating the shell 158, the cooperating collar walls 152 and 154 can form a mechanical connection using frictional engagement to securely hold the control module 104 against the dilator module 102. Although FIGS. 5A-5C depict connection assemblies for engaging the dilator module 102 with the control module 104 that uses a set of rotating collar walls that form a mechanical engagement and frictional bond between the two modules, it will be apparent to those of skill and the art that other assembly mechanisms may be employed for joining the expansion module 104 to the dilator module 102 without departing from the scope hereof. Such alternatives may include exterior latches that a clinician may use to latch the expansion module 104 to the dilator module 102, exterior threaded collars that can slide over the expansion module 104 and dilator module 102 each of which modules may have a threaded exterior ring and the exterior threaded collar may rotate about the threaded rings of the dilator module 102 and the control module 104 to cause the two modules 102 and 1042 to be securely joined together. These and other embodiments may be employed without departing from the scope hereof.

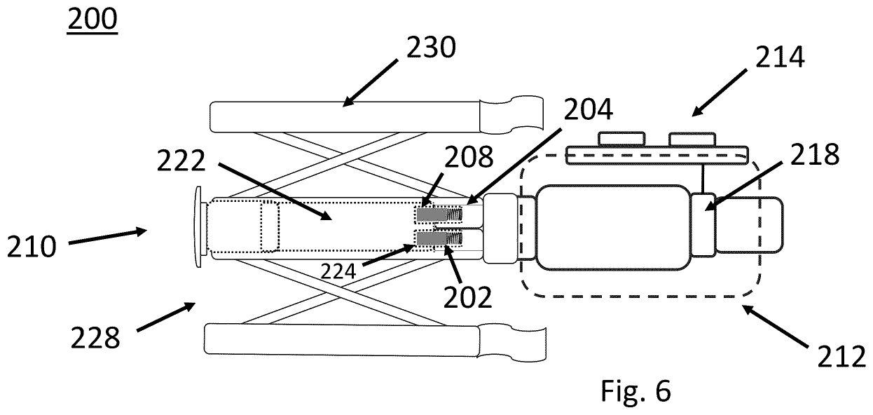

FIG. 6 depicts in schematic form and with a cut away view a medical device 200 of the type described herein and shows in more detail one embodiment for connecting a scissor mechanism of a dilator module to a rotating spindle of an control module. In particular, FIG. 6 depicts a device 200 that includes a dilator module 210, an control module 212, a user interface 214, a motor control assembly 218, springs 202, a spindle 204, detents 208, recesses 224 and a lead screw 222. In the embodiment of FIG. 6 a rotatable lead screw 222 of a scissor assembly 228 connects by a releasable mechanical connection of spring driven detents 208 in a spindle 204 of a dilator module 210. Complementary recesses 224 in the lead screw 222 receive the detents 208 to join the spindle 204 to the lead screw 222 in a manner that allows for the transfer of a torque from the spindle 204 to the lead screw 222.

In the embodiment of FIG. 6 the control module 212 includes a spindle 204 that extends distally from the expansion and control module 212. At the distal end of the spindle 204 are two spring loaded detents 208. Each detent 208 is connected to a spring 202 that drives the detent 208 proximally into a complementary recess 224 within the lead screw 222. The depicted lead screw 222 has two recesses 224 that are sized and located to receive the detents 208. In other embodiments, there can be more recesses and detents. The recesses and detents of this embodiment are generally cylindrical, but the recesses and detents may have any suitable shape, including cubes. Further, although the spring driven detents 208 are, in this embodiment, located in the spindle, in other embodiments, the spring-loaded detents may be part of the lead screw and the complementary recesses may be located in the spindle 204. The depicted detents 208 may be metal, such as aluminum or plastic or some other suitable material, but in any case the detents 208 are configured to provide sufficient shear strength, to deliver a torque from the rotating spindle 224 to the screw 222. Optionally, the detents may have a set shear factor to act as shear pins which prevent the transmission of a torque over a predefined shear limit for the detent.

In the depicted embodiment, when the control module 212 is rotatably engaged to the dilator module 210, as discussed above with respect to FIGS. 5A-5C, the detents 208 will align with the recesses 224 and by action of springs 202 drive into the recesses 224. Once within the recesses 224 the control module 212 can drive the screw 222 to expand or contract the scissor mechanism which drives out or pulls in, depending on the direction of rotation of the screw, the padded arms 230.

Although FIG. 6 depicts an embodiment that uses spring loaded detents or pins to mechanically couple a spindle to a rotating screw in a manner that will transfer a torque, it will be understood that in other embodiments other mechanical couplings for joining a spindle to an element of the expanding dilator module may be employed. For example, in alternate embodiments, magnets may be used to join the spindle to the lead screw by having a magnetic attachment between the spindle and the lead screw. In such an embodiment, both the lead screw and the spindle may include magnets, such as neodymium magnets of the type used in medical devices. The magnets in the lead screw may be arranged to attract the magnets in the spindle. As the dilator module is joined to the control module, the magnetic attraction between the magnets in the spindle and the magnets in the lead screw can cause a mechanical connection sufficient to transfer a torque from the spindle to the lead screw. In still other embodiments, where the dilator module expands from a contracted configuration to an expanded configuration by means other than a scissor mechanism, such as by a hydraulic force, other mechanisms may be employed such as fluid coupling connections that allow a fluid driven from the control module to be received by the dilator module which will respond to the hydraulic force by expanding or contracting the padded arms of the dilator.

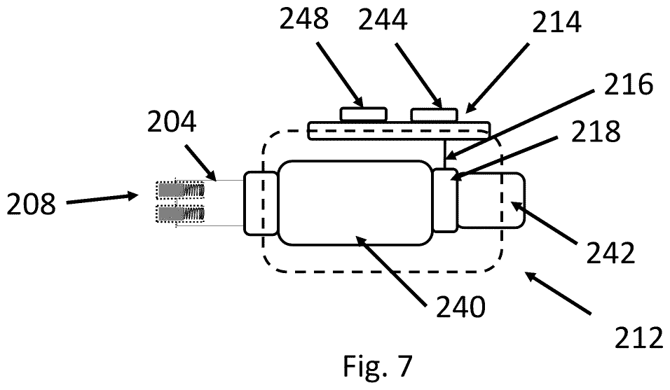

FIG. 7 depicts a partial cutaway view of the control module 212 of FIG. 6, showing it separate from the dilator module 210. In the embodiment of FIG. 7, the control module is configured to both control the dilator module and drive the expansion and contraction of the dilator module. As such, the control module 212, which is also in the depicted embodiment shaped like a handle that the clinician can manipulate, integrates user interface controls along with the motor assembly that will drive the lead screw of the scissor mechanism in the dilator module. It will be understood that in alternative embodiments, the device 200 may include a control module that lacks the control features such as the user interface 214 of the depicted control module 212. In one such alternate embodiment, the user interface 214 may be replaced with a wireless transceiver system, typically a Bluetooth system, that will allow a clinician to employ a separate processing system for sending commands to the control module in this embodiment. Typically the Bluetooth transceiver built into the control module of this alternative embodiment will communicate with an application (“an app”) running on the clinician′ mobile device, such as a cell phone, or the patient's cell phone. The app can provide user interface features that can be manipulated to cause the control module to carefully and therapeutically cause expansion and contraction of the dilator module for therapeutic treatment of the patient.

Returning to the embodiment of FIG. 7, the depicted control module 212 includes a user interface 214 with components for allowing the clinician to control the operation of the motor 240. In this embodiment the user interface 214 will allow the user to manually control the expansion and contraction of the dilator module by manipulating the user interface controls such as the control buttons 244 and 248. The control module 212 includes a motor 240, a motor control assembly 218, and a removable battery 242. The depicted user interface 214 includes a first control button 244 and a second control button 248. As shown in FIG. 7 the user interface 214 connects to the motor control assembly 218 via communication path 216, which is this embodiment is a serial data path for carrying electrical signals back and forth between the user interface 214 and the motor control assembly 218. The clinician using the user interface 214 by activating the buttons 244 and 248 can cause the motor control assembly 218 to drive the spindle 204 in a manner that will expand or contract the dilation module 210. In one embodiment one control button 244 is used to expand the dilator module 210 and the other control button 248 is used to direct contraction of the dilator module 210. Optionally, the user interface 214 may also include display elements, such as configurable light emitting diodes. The motor control assembly 218 may pass signals across the communication path 216 that the user interface 214 can process. Such signals may be used by the user interface 214 to activate the display elements and thereby pass status information to the clinician.

In the embodiment depicted in FIG. 7 the motor control assembly 218 includes a programmable motor control circuit of the type that includes a microcontroller. The motor control assembly 218 monitors the data path 216 between the motor control assembly 218 and the user interface 24. The motor control assembly 218 can respond to activation of either control button 244 and 248 and carry out a programmatic therapeutic expansion, or contraction of the dilator module 210 in response to the clinician having activated either of the controls 244 or 248. For example, in response to an activation of a control button such as the button 244 that directs the motor assembly 218 to expand the dilator module 210 from a contracted configuration, the programmable motor control assembly 218 may use the microcontroller to execute a programmed set of steps that incrementally and over a set period of time such as 30 minutes, 45 minutes, an hour, an hour and a half or more, may cause the motor control assembly 218 to control the motor 240 to apply a torque to the spindle 204 to drive expansion of the dilator module 210. Typically, a force sensor (not shown) is provided that determines the force being applied by the dilator module 210 to the tissue wall of the patient. The force sensor can provide information that can be sent to the programmable motor control assembly 218. The motor control assembly 218 can control the applied force and if the applied force exceeds a threshold value of force, the motor control assembly 218 can, in one practice, stop expansion, and in another practice, stop expansion and contract the dilator module 210. Such reversal can be an incremental decrement in the radius of the dilation module 210 to reduce the amount of pressure or force applied to the tissue wall of the patient. In one embodiment, the motor control assembly 218 determines the force being applied to the tissue of the patient by monitoring the current demand being drawn by the motor 240. Those of skill in the art will know that there are numerous techniques for measuring and analyzing voltage and power changes at the input to a motor to determine the load being applied by the motor. This load is representative of the force ultimately being applied by the dilator module 210 to the tissue of the patient. As such the programmable motor control assembly can measure the current and force applied to the motor 240 and from those measurements calculate a measure of the force being applied to the tissue of the patient. Optionally, in alternate embodiments the spindle 204 may also include an electrical connection that can connect to an electrical connection carried within the screw 230 of the dilator module 210. Force sensors of the type that measure a force applied can be connected to the pads 238 of the dilator module 210. The force sensors can provide measures of the force being applied to the tissue wall and can transform these measures into electrical signals that can be transferred through the electrical connectors in the screw 238 and the spindle 204. In this way the force sensors in pads 238 carried on the dilator module 210 can be electrically connected through the spindle 204 to the programmable motor control assembly 218. The programmable motor control assembly 218 may monitor the signals from the force sensors located on the padded arms 238 and compare the measured forces to a threshold force or forces and determine whether to stop incrementing the diameter of the dilator module 210 an optionally whether to reduce the diameter of the dilator module 210.

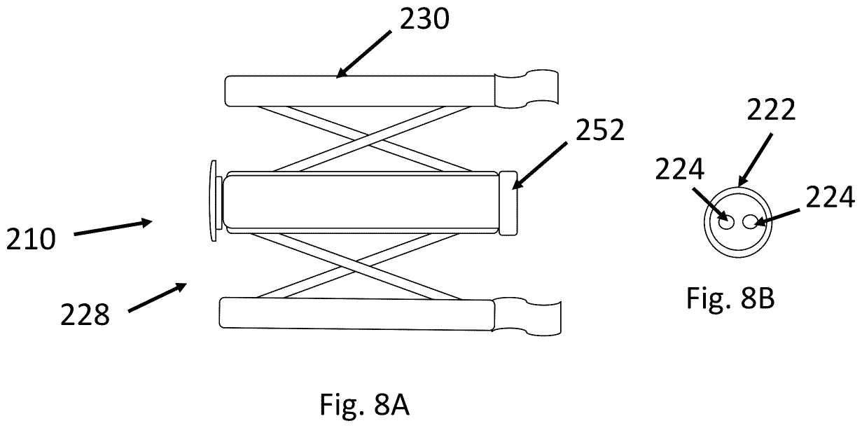

FIGS. 8A and 8B depict in more detail certain components of the dilator module 210. In particular FIG. 8A depicts the scissor mechanism 228 that moves the padded arms 230 radially away from the longitudinal axis 106 that extends through the center of the dilator module 210. Dilator module 210 includes a collar 252 that can receive the control 212. FIG. 8B depicts from a planar perspective the face of the screw 222 and in particular shows the two recesses 224 each placed within the proximal face of the screw 222, which opposes the control module 210 and interfaces with the detents of the spindle 204.

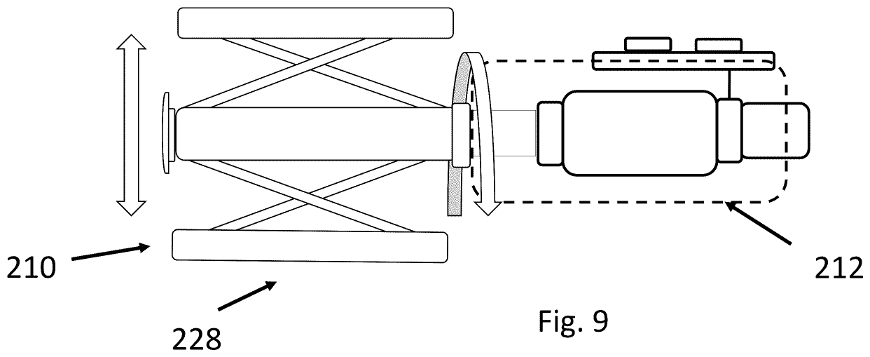

FIG. 9 depicts the assembled device 200 with the dilator module 210 connected to and being driven by the control module 212. As depicted in FIG. 9, in operation the spindle rotates to provide a torque force that drives the padded arms of the scissor mechanism 228 radially away from the longitudinal access 106. This allows for the radial expansion of the arms as shown in FIG. 9. As shown in FIG. 1C, once the device is expanded, the control module may be separated from the dilator module 210. The dilator module 210 may be left within the patient for the purpose of continuing the therapy and such therapies can take 30 minutes to 20 minutes of time with increments of expansion happening every minute or two. But more commonly the control module 212 is maintained in contact with and in connection with the dilator module 210 throughout the procedure of preparing the tissue for delivery. After the procedure, the dilator module 210 can be disconnected from the expansion and control module 212 and the dilator module 210 may be disposed of. The control module 212 may be saved and reused in a subsequent procedure with another patient. Typically, this involves a sterilization process that allows the control module 212 to be reused.

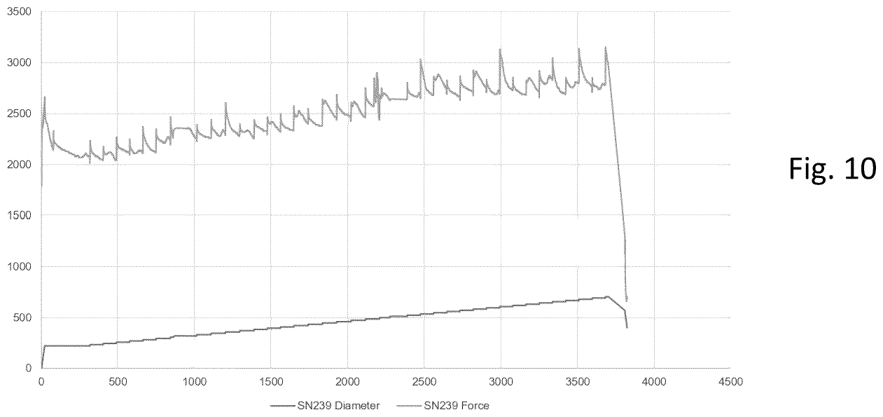

FIG. 10 depicts one process for using the devices described herein for treating a patient prior to delivery. As depicted in FIG. 10 one sees a graph that represents the force being applied to the tissue wall of the patient, the diameter of the patient's vaginal canal during those times and shows a treatment that is undertaken for approximately 3600 seconds, or one hour (as measured along the X-axis). During this treatment the diameter of the device increases from about 2.5 cm to about 7.5 cm. This is done by applying a force which is shown as a measure along the Y-axis. As depicted in FIG. 10 for this particular procedure a relatively small incremental increase in force is applied approximately every three or four minutes during which time the control module of the device will cause the dilator module to incrementally increase in radius and then will hold the dilator module at that radius for a period of time, such as a few minutes, to allow the tissue to relax and expand comfortably and reduction of the likelihood of harm to the patient. This continues as radial expansion goes forward from, as shown in FIG. 10 about 2.5 cm to about 8 cm. For the procedure depicted in FIG. 10 the procedure is carried out for about 3600 seconds which is about a 60 minute time period of treatment.

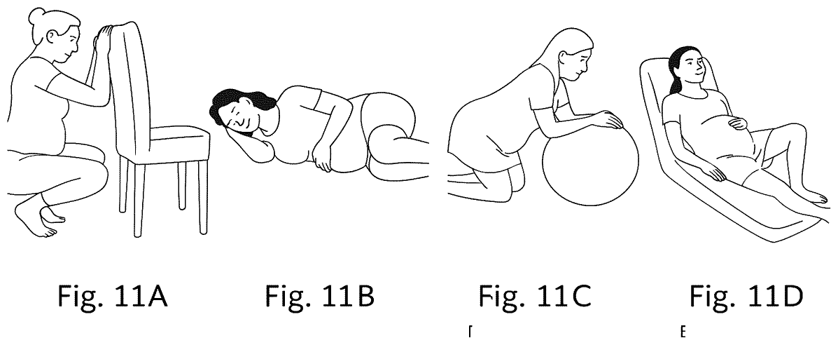

FIG. 10 depicts a continuous process that begins with insertion of the device, such as device 200, into the patient until dilation is complete and the device is removed by the clinician from the patient. However, in alternate procedures, the clinician may pause dilation for the purpose of allowing the patient to comfortably and more easily move to a different location or to reposition themselves, as well as to conduct certain exercises for the purposes of improving comfort during the labor and delivery process. FIGS. 11A through 11D depict mothers going through the process of Labor and delivery and positioning themselves for improved comfort and delivery. In one practice of the methods described herein, a clinician may remove the control module from the device to reduce the size, and weight of the device and thereby make it easier for the patient to reposition themselves and move about a room going through the common movements of Labor depicted in FIGS. 11A through 11D. It will be understood that by removing the control module from the device, the weight of the device is reduced. This reduction in weight reduces the likelihood the device will fall from the patient as the patient moves through the common movements of Labor such as those depicted in FIGS. 11A through 11D. As a patient may want to spend more than 15 minutes moving through these positions of Labor, the process will pause at a set dilation diameter while the control module is removed from the device. This would appear in FIG. 10 as a period of time, such as 15 minutes or half an hour depending upon the time period taken by the patient for this repositioning process, during which no expansion takes place and the dilator module is held at a fixed diameter.

To facilitate such a process, the control module may detect when it has been disconnected from the dilator module. The control module can store information about the current state of the device including the time period at which the control module was disconnected from the dilator, the diameter of the dilator, and the time period that had been set for the dilation procedure, such as 60 minutes. Upon reconnecting the control module to the dilator module, the motor control assembly may begin the procedure of dilation from the point at which the procedure had stopped due to the disconnection of the expansion and control module. Alternatively, the control module may begin a slightly accelerated dilation process in order to keep the patient on track for full dilation within an hour to an hour and a half of beginning treatment. In this way, the control module targets completion of dilation for a time period close to when one expects the cervix to be fully open and descent of the baby to begin.

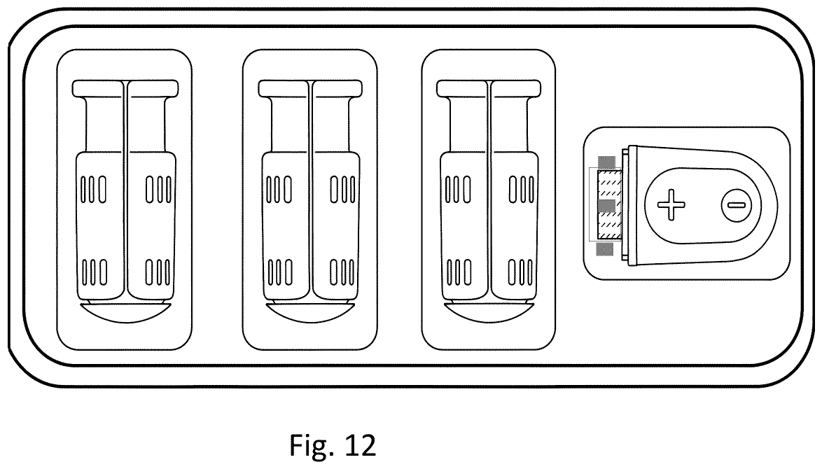

After treatment, the device 200 is removed from the patient by the clinician. As discussed with reference to FIGS. 5A through 5C, the clinician or technician may separate the dilator module 210 from the expansion and control module 212. The dilator module 210 may be disposed of as waste. The control module 212 may be sterilized and reused. In one aspect the devices described herein may include a kit that includes one actuation module, such as a control module, and several dilator modules. FIG. 12 depicts one example of such a kit. FIG. 12 depicts a container holding three sterilized dilator modules and a single actuation module. The dilator modules are disposable and the actuator module may be used for multiple procedures, typically going through a conventional sterilization process between procedures.

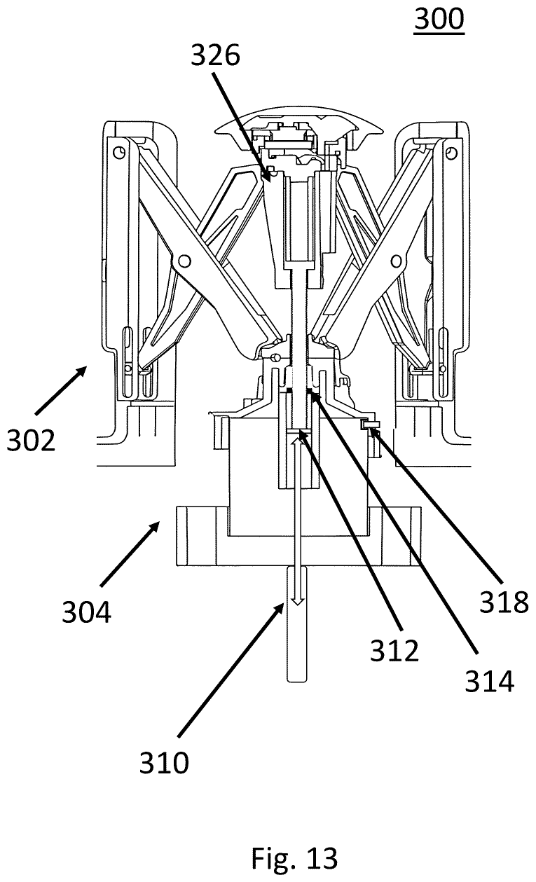

FIG. 13 depicts an alternative embodiment of the systems and methods described herein. FIG. 13 depicts a device 300 of the type described herein and wherein the releasable mechanical coupling comprises a spring loaded pin 318 and recess of the type commonly used for mechanically pinning one element of a mechanical device in place relative to another element of that mechanical device. In this example, the pin of the pin and recess 318 extends through a housing of the dilator module 302 and into a recess of the housing of the control module 304. The spring loaded pin can be released by manually lifting the pin away from the dilator module 302 such that the pin is lifted from the recess in the control module, thereby leaving the control module 304 capable of being moved axially away from the dilator module 302. The control module 304 includes a fluidic coupling 310 for delivering a fluid under pressure (either high pressure or vacuum pressure) to the dilator module 302 to control expansion and contraction of the dilator module 302. In this embodiment, the control module 304 includes a fluidic coupling 310 that can deliver a fluid, typically air, and that air may be delivered to the piston 312 that connects to the sled 326 of the dilated module 302. A seal 314 maintains pressure within the fluidic coupling area such that positive air pressure can provide a force for moving the piston 312 forward to cause the sled 326 to move forward and the dilator module 302 to contract. Similarly, a vacuum (negative pressure) applied through fluidic coupling 310 can draw the piston 312 proximally, thereby causing radial expansion of the dilated module 302. Although the embodiment of FIG. 13 is discussed with application of air pressure or vacuum, it'll be understood by those of skill in the art that other hydraulic forces including hydraulic forces by application of water, saline or hydraulic fluid, can be used without departing from the scope hereof.

Although most embodiments described herein show the arms as a scissor-like assembly, it should be understood that other methods and apparatus for expanding the pads can be used. For example, the arms can be singular arms attached to the pads (e.g., similar to a speculum).

The vaginal dilation device can be sized, shaped, and configured to penetrate approximately the first third, or 3-4 cm, of the vagina, and to gradually expand the vaginal introitus from a resting diameter of approximately 2 cm to a fully dilated diameter of approximately 10 cm. The vaginal dilation device can be configured to expand from a compact, closed configuration, as shown in FIG. 1A, to an expanded configuration, as shown in FIGS. 1B and C. When the device is in the contracted configuration, the pads can be seamlessly closed against each adjacent pad so as to form a solid shape (e.g., circle, oval, etc.).

When the device is in the closed configuration the padded arms can be configured to rest against each adjacent pad so as to reduce the outer diameter of the device. In some embodiments, the diameter of device at the position of the padded arms in the closed configuration can be less than 4 cm. In one embodiment, the outer diameter of the pads in the closed configuration is approximately 2-4 cm.

As the device dilates to the expanded configuration, as shown in FIGS. 1B-1C, the padded arms move radially outwards from the device, causing the pads to separate from one another. In some embodiments, the maximum diameter of the pads in the expanded configuration can be approximately 10 cm. In one embodiment, the maximum outer diameter of the pads in the expanded configuration is approximately 8-12 cm.

The vaginal dilation device 100 is specifically designed to promote compactness, effective dilation, and good tissue contact during expansion. As the device expands from the closed configuration to the expanded configuration, the center of mass of the device can move under the pads which helps keep the device in place without rotating and falling out of the vagina.

The dilation device can further comprise indicators or gauges. In some embodiments, the gauge gives the user an indication of the diameter of the padded arms. A user, such as a physician, can then use the gauge to know the exact amount of tissue dilation. Gauges can comprise a simple binary readout (showing the diameter is above or below some threshold to continue dilating), or can have a scale showing the actual diameter of the device, for example.

The dilation device 200 may additionally include a quick-release mechanism configured to collapse the device from the expanded configuration to the closed configuration. The quick-release mechanism can comprise distal contact plate 140. Additional details regarding such a quick-release mechanism are found in U.S. Pat. No. 9,492,197 assigned to the assignee hereof.

In other embodiments, the device can include an alarm or alert mechanism, such as a visual alert (e.g., a light, or a warning indicator on a display) or an audible alert (e.g., a buzzer or an alarm sound) to indicate to a user that the device is applying too much, or too little force to the vagina. The alert mechanism can also include a timer configured to alert the user (e.g., by an audible or visual signal) when to dilate the device.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A device for dilating a vaginal canal of a patient during labor, comprising:
a dilator module having a longitudinal axis and being sized to fit in a vaginal canal to traverse the vaginal introitus and extend to a position inferior to the cervix, and having an expandable padded exterior configured to expand radially from a contracted configuration having a first diameter to an expanded configuration having a second larger diameter;
a control module configured to cause the dilator module to expand the padded exterior from the contracted configuration to the expanded configuration, and
a releasable mechanical coupling configured to releasably connect the control module to the dilation module.

2. The device of claim 1, wherein the control module comprises
a programmable controller configured to control the control module to expand the padded exterior to contact a tissue wall of the vaginal canal of the patient.

3. The device of claim 2, further comprising
a force sensor capable of measuring a force applied by the padded exterior to the tissue wall of the vaginal canal of the patient and communicating with the programmable controller.

4. The device of claim 1, wherein the releasable mechanical coupling includes a coupling for delivering a torque to the dilator module to control expansion and contraction of the dilator module.

5. The device of claim 1, wherein the releasable mechanical coupling includes a fluidic coupling for delivering a fluid under pressure to the dilator module to control expansion and contraction of the dilator module.

6. The device of claim 1, wherein the handle has a length along the longitudinal axis selected to extend from the vaginal canal of the patient for a length that is sufficiently short to allow the patient to comfortably carry the device during the typical movements of labor.

7. The device of claim 1, wherein the handle has a length along the longitudinal axis of between 4 cm and 20 cm.

8. The device of claim 1, wherein the handle has a diameter sized to be manually gripped by treating clinician.

9. The device of claim 1, wherein the handle has a diameter sized between 2 cm and 10 cm.

10. The device of claim 1 wherein the controller includes a processor for implementing a program to automatically expand the dilator module.

11. The device of claim 1 wherein the dilator module releasably connects to the control module to align along the longitudinal axis such that a linear force applied to the handle will act to move the dilator module along the longitudinal axis for insertion into the vaginal canal of the patient.

12. A method for dilating a vaginal canal of a patient during labor, comprising:
providing a dilator module having a longitudinal axis and being sized to fit in a vaginal canal to traverse the vaginal introitus and extend to a position inferior to the cervix, and having an expandable padded exterior configured to expand radially from a contracted configuration having a first diameter to an expanded configuration having a second larger diameter;
providing an control module configured to cause the dilator module to expand the padded exterior from the contracted configuration to the expanded configuration,
providing a releasable mechanical coupling configured to releasably connect the control module to the dilator module,
releasably connecting the dilator module to the control module to align along the longitudinal axis.

13. The method of claim 12 further including applying a linear force to the handle to move the dilator module along the longitudinal axis for insertion into the vaginal canal of the patient.

14. The method of claim 12, further comprising controlling the control module to expand the padded exterior to contact a tissue wall of the vaginal canal of the patient.

15. The method of claim 12 wherein controlling includes implementing a program to automatically expand the dilator module to therapeutically treat tissue of a vaginal canal during a first stage of labor.

16. The method of claim 15, wherein controlling includes pausing the program of automatically expanding.

17. The method of claim 12, further include operating the releasable mechanical coupling to release the control module from the dilator module during stage one of labor.

18. The method of claim 12, wherein releasably connecting the dilator module to the control module includes coupling a drive shaft of the control module to a drive coupling of the dilator.

19. The method of claim 12, wherein releasably connecting the dilator module to the control module includes coupling a fluidic coupling to the dilator.

20. A method of manufacturing a device for dilating a vaginal canal of a patient during labor, comprising:
providing a dilator module having a longitudinal axis and being sized to fit in a vaginal canal to traverse the vaginal introitus and extend to a position inferior to the cervix, and having an expandable exterior configured to expand radially from a contracted configuration having a first diameter to an expanded configuration having a second larger diameter;
providing an control module configured to cause the dilator module to expand the exterior from the contracted configuration to the expanded configuration, and
attaching to at least one of the control module and the dilator module, a releasable mechanical coupling configured to releasably connect the control module to the dilator module.
